(19) United States
(12) Patent Application Publication
(10) Pub. No.: US 2014/0275166 A1
(43) Pub. Date: Sep. 18, 2014

(54) SPINTRAPS AND SPIN LABELS AND THEIR HYDROXYLAMINES FOR PERI- AND POST-MENOPAUSAL SYNDROME

(71) Applicant: Peter Herbert Proctor, Houston, TX (US)

(72) Inventor: Peter Herbert Proctor, Houston, TX (US)

(21) Appl. No.: 14/197,524

(22) Filed: Mar. 5, 2014

Related U.S. Application Data
(60) Provisional application No. 61/852,385, filed on Mar. 15, 2013.

(51) Int. Cl. A61K 31/445; A61K 31/133; A61K 31/15

(52) U.S. Cl. CPC A61K 31/445; A61K 31/15; A61K 31/133
USPC 514/315; 514/640; 514/645

(57) ABSTRACT

Spin-traps and spin labels and their reduction products are claimed for the treatment of peri- and post-menopausal syndrome.

Priority is claimed under provision patent application 61/852,385 filed Mar. 15, 2013

### BACKGROUND OF THE INVENTION

(1) Field of Invention

Treatment of Peri- and Post-menopausal syndrome

(2) Description of Related Art

### Citations

Cuscela, Daniel, et al. “Protection from Radiation-Induced Alopecia with Topical Application of Nitroxides: Fractionated Studies”, The Cancer Journal from Scientific American, 1996; vol. 2, No. 5, pp. 273-278.

Enli Y, Oztekin O, Pinarbasili R D. The nitroxide tempol has similar antioxidant effects as physiological levels of 17 beta-oestradiol in reversing ovariectomy-induced oxidative stress in mice liver and kidney. Scand J Clin Lab Invest. 2009;69(4):526-34).

Floyd R A. Serendipitous findings while researching oxygen free radicals. Free Radic Biol Med. 2009 Apr. 15;46(8):1004-13.

Janzen E G., “Spin trapping and associated vocabulary”, Free Rad Res Comm 1990;9(3-6):163,

Kang L S, Chen B, Reyes R A, Leblanc A J, Teng B, Mustafa S J, Muller-Delp J M. Aging and estrogen alter endothelial reactivity to reactive oxygen species in coronary arterioles. Am J Physiol Heart Circ Physiol. 2011 June;300(6):H2105-15

Kamat C D, Gadal S, Mhatre M, Williamson K S, Pye Q N, Hensley K. Antioxidants in central nervous system diseases: preclinical promise and translational challenges. J Alzheimers Dis. 2008 November;15(3):473-93.

Mitchell, B A, Menopausal Hormone Therapy for the Primary Prevention of Chronic Conditions: A Systematic Review to Update the U.S. Preventive Services Task Force Recommendations, Ann Intern Med. (2012);157(2):104-113.

Nelson, H D; Miranda Walker, MA; Bernadette Zakher, MBBS; and Jennifer Pitkin J. Alternative and complementary therapies for the menopause. Menopause Int. 2012 March;18(1):20-7.

PAC, 1994, 66, 1077 (Glossary of terms used in physical organic chemistry (IUPAC Recommendations 1994)) page 1166.

Proctor P H, Tamborello L P. SAINT-I worked, but the neuroprotectant is not NXY-059. Stroke. 2007 October;38(10):e109

Proctor P H. Uric acid and neuroprotection. Stroke. 2008 August;39(8):e126.Augusto O,

Trindade D F, Linares E, Vaz S M. Cyclic nitroxides inhibit the toxicity of nitric oxide-derived oxidants: mechanisms and implications. An Acad Bras Cienc. 2008 March;80(1):179-89.

Reslan, O M and Raouf A. Khalil, Vascular Effects of Estrogenic Menopausal Hormone Therapy, Rev Recent Clin Trials. 2012 Feb. 1; 7(1): 47-70.

Santen R J, Mansel R. Benign breast disorders. N Engl J Med. 2005;353(3):275-85. Review.

Sartori-Valinotti J C, Iliescu R, Fortepiani L A, Yanes L L, and Reckelhoff J F (2007), Sex differences in oxidative stress and the impact on blood pressure control and cardiovascular disease. Clin Exp Pharmacol Physiol 34: 938-945.

Shuaib A, Lees K R, Lyden P, Grotta J, Davalos A, Davis S M, Diener H C, Ashwood T, Wasiewski W W, Emeribe U. NXY-059 for the treatment of acute ischemic stroke. N Engl J Med. 2007; 357: 562-571

Simonsen U, Christensen F H, Buus N H. The effect of tempol on endothelium-dependent vasodilatation and blood pressure. Pharmacol Ther. 2009 May;122(2):109-24.

Velarde M C. Pleiotropic actions of estrogen: a mitochondrial matter. Physiol Genomics. 2013 Feb. 4;45(3):106-9.

Wilcox C S. Effects of tempol and redox-cycling nitroxides in models of oxidative stress. Pharmacol Ther. 2010 May;126(2):119-45,

Wilcox C S, Pearlman A. Chemistry and antihypertensive effects of tempol and other nitroxides. Pharmacol Rev. 2008 December;60(4):418-69.

Various publications, including patents, published applications, and scholarly or technical articles are cited above and throughout the specification. Each of the cited publications is incorporated by reference herein, in its entirety.

### Field OF THE INVENTION

In women, menopause is brought on by cessation of production of estriol and progestins by the ovaries. This happens naturally about age 50 with cessation of menses, but also occurs in the treatment of breast cancer and ovarian cancer with antiestrogens or ovariectomy. Peri- and post-menopausal syndrome is a chronic potentially-debilitating condition associated with significant morbidity, especially discomfort associated with “hot flashes”, with a feeling of heat, diaphoresis, sleep-disturbance, changes in mood (including depression), dermal changes, and general discomfort. Presently, the main treatment is “hormone replacement”, typically with combinations of estrogens and progestins (Pitkin J. Alternative and complementary therapies for the menopause. Menopause Int. 2012 March;18(1):20-7). Unfortunately, “hormone replacement therapy” (HRT) is associated with an increased hazard of breast cancer, heart attack, and stroke (Heidi D. Nelson, MD, MPH; Miranda Walker, MA; Bernadette Zakher, MBBS; and Jennifer Mitchell, BA, Menopausal Hormone Therapy for the Primary Prevention of Chronic Conditions: A Systematic Review to Update the U.S. Preventive Services Task Force Recommendations, Ann Intern Med.(2012);157(2):104-113). The various and complicated effects of estrogens are particularly important, particularly with respect to the vasculature (Ossama M. Reslan and Raouf A. Khalil, Vascular Effects of Estrogenic Menopausal Hormone Therapy, Rev Recent Clin Trials. 2012 Feb. 1; 7(1): 47-70, Velarde M C. Pleiotropic actions of estrogen: a mitochondrial matter. Physiol Genomics. 2013 Feb. 4;45(3):106-9.)

The efficacy of various aminoxyl-forming spin traps and aminoxyl spin labels and their equivalent reduction products in some model diseases in experimental animals has long been recognized. For reviews, see: Wilcox C S. (Effects of tempol and redox-cycling nitroxides in models of oxidative stress. Pharmacol Ther. 2010 May;126(2):119-45), Wilcox C S, and Pearlman A. (Chemistry and antihypertensive effects of tempol and other nitroxides. Pharmacol Rev. 2008 December;60(4):418-69), Simonsen U, et al. (The effect of tempol on endothelium-dependent vasodilatation and blood pressure. Pharmacol Ther. 2009 May;122(2):109-24., and Floyd R A., Serendipitous findings while researching oxygen free radicals. Free Radic Biol Med. 2009 Apr. 15;46(8):1004-13, emphasis-added).

However, studies with such antioxidant drugs have not well translated to humans. E.g., with respect to neuroprotection and cardioprotection in general and nitrone and nitroxide drugs in particular: “There are hundreds, perhaps thousands of neuroprotective drugs that have been used in animal models. So, if you were a mouse or a rat, and experienced a stroke or cardiac arrest, we would know just what to do for you. But, essentially none of these pharmacological agents have demonstrated usefulness in humans even though they have been shown to be successful in preclinical animal trials ” (Traystman R J. Neuroprotection: introduction. Stroke. 2010 October;41(10 Suppl):563.). In particular, one such neuroprotective agent effective in animal models, but not in humans, is the phenybutylnitrone derivative NXY-059 (Shuaib A, Lees K R, Lyden P, Grotta J, Davalos A, Davis S M, Diener H C, Ashwood T, Wasiewski W W, Emeribe U. NXY-059 for the treatment of acute ischemic stroke. N Engl J Med. 2007; 357: 562-571). In turn, the spin-trap methylnitrosopropane (MNP) is likely the pharmacologically-active breakdown product of NXY-059 and other phenylbutylnitrone derivatives. (Proctor P H, Tamborello L P. SAINT-I worked, but the neuroprotectant is not NXY-059. Stroke. 2007 October;38(10):e109)

Moreover, in animals treatment effects are reported at systemic human equivalent doses orders of magnitude higher than we found efficacy in (e.g.) perimenstrual syndrome in humans. If the in vivo active form is (say) the reduced derivative, this may be an unexpected consequence of the presence in humans and higher primates of singularly high levels of the powerful reducing substance uric acid. Paradoxically, since it competes with them for action, the singularly-high level of urate in humans may also may explain the perpetual failure of antioxidant drugs such as NXY-059 in human clinical trials, even at several grams per day doses (Proctor P H. Uric acid and neuroprotection. Stroke. 2008 August;39(8):e126.). That is, any systemic efficacy of such aminoxyl and aminoxyl-forming drugs in humans, much less efficacy at very low doses, is unexpected. Similarly, we recognized the potential benefit of administering such compounds topically in their reduced forms (U.S. Pat. Nos. 5,714,482, 5,714,510, 5,716,947, 5,723,502, 5,728,714), e.g., in presence of a strong reducing compound such as ascorbic acid. For one thing, coadministration in the reduced form or in the presence of a reducing substances may prevent depletion of endogenous reducing equivalents by the parent drugs. These patents are here-by incorporated by reference. However, the efficacy, much less the benefits, of the using such reduced forms is disputed. E.g., Hsia et al (U.S. Pat. No. 7,314,633) note that “If a nitroxide is reduced to a hydroxylamine it loses its ability to modulate reactions. By positioning the nitroxide between two carboxylic acid groups a “gating” effect is obtained, i.e. the nitroxide is protected and its ability to modulate reactions is maintained over a longer period of time in a greater range of in vivo environments as compared to a molecule lacking the carboxylic acid groups.” Previously, the only human use of (e.g.) TEMPOL was experimental topical treatment to ameliorate radiation injury (Cuscela, Daniel, et al. “Protection from Radiation-Induced Alopecia with Topical Application of Nitroxides: Fractionated Studies”, The Cancer Journal from Scientific American, 1996; vol. 2, No. 5, pp. 273-278.). In ovariectomized mouse liver and kidney, TEMPOL is reported to have similar antioxidant effects to estrogen (Enli Y, Oztekin O, Pinarbasili R D. The nitroxide tempol has similar antioxidant effects as physiological levels of 17 beta-oestradiol in reversing ovariectomy-induced oxidative stress in mice liver and kidney. Scand J Clin Lab Invest. 2009;69(4):526-34). However, antioxidants in general do not reverse the symptoms of menopause, nor is TEMPOL-reported to have an estrogen-like effect otherwise. So this does not teach the use of TEMPOL to treat perimenopausal symptoms in humans. Further, the scientific literature positively teaches against the use of TEMPOL and similar compounds in the treatment of peri and post-menopausal syndrome and notes the variability of the treatment response with respect to estrogen status and animal models for menopause produced by ovariectomy (“OVE”).

For example, Wilcox and Perlman (Wilcox C S, Pearlman A. Chemistry and antihypertensive effects of tempol and other nitroxides. Pharmacol Rev. 2008 December;60(4):418-69.) note that “A surprising finding has been the variable BP response to tempol in female SHR (citing Sartori-Valinotti J C, Iliescu R, Fortepiani L A, Yanes L L, and Reckelhoff J F (2007), Sex differences in oxidative stress and the impact on blood pressure control and cardiovascular disease. Clin Exp Pharmacol Physiol 34: 938-945.). Emphasis-added, where “SHR” means “Spontaneously hypertensive rats”.

Likewise, whereas male SHR had an antihypertensive response to tempol as did female SHR administered tempol for the first 15 weeks of their life (Fortepiani A and Reckelhoff J F (2005) Role of oxidative stress in the sex differences in blood pressure in spontaneously hypertensive rats. J Hypertens 23: 801-805.), “no response was observed in postmenopausal female SHR” (Fortepiani L A, Zhang H, Racusen L, Roberts L J 2nd, and Reckelhoff J F (2003) Characterization of an animal model of postmenopausal hypertension in spontaneously hypertensive rats. Hypertension 41: 640-645.)

Likewise “. . . Remarkably, tempol was more effective in lowering the BP of young female than young male SHR and became less effective after menopause. The finding that aged, postmenopausal female SHR, which lack estrogen, would lack an antihypertensive response to tempol was unexpected because other observations by this group attested to increased ROS generation in postmenopausal rats (Fortepiani et al., 2003).” Similarly, Kang et al (Lori S. Kang, Bei Chen, Rafael A. Reyes, Amanda J. LeBlanc, Bunyen Teng, S. Jamal Mustafa, and Judy M. Muller-Del, Aging and estrogen alter endothelial reactivity to reactive oxygen species in coronary arterioles, Am J Physiol Heart Circ Physiol. 2011; 300(6): H2105-H2115.) report a similar unexpected lack of effect of TEMPOL on vascular responsiveness in ovariectomized rats. Sic: “Contrary to our expectations, we found that the O2—scavenger Tempol blunted flow-induced increases in NO and subsequent vasodilation in arterioles from intact and OVE females”.

TEMPOL-H is also effective in the treatment of fibrocystic disease of breast (US20120115905), in which estrogens and progestins play a role. This would steer one skilled in the art away from using this compound to treat perimenopausal syndrome, where the exact opposite, estrogen and progestin deficiency, produces the signs and symptoms of the disorder.

In sum, all not only outline the unpredictability of the actions of the claimed agents, but also teach specifically against any potential action in the treatment of perimenopausal syndrome.

### BRIEF SUMMARY OF THE INVENTION

The present invention is directed to methods that treat, inhibit, or slow the development of the signs and symptoms of menopause. The inventive methods comprise the administration of pharmaceutical preparations comprising spin traps and spin labels.

### DESCRIPTION OF THE INVENTION

Definitions:

Jansen defines “spin trap”: “Spintrapping is an anlytical technique for detecting short-lived reactive free radicals in biological systems by providing a nitrone or nitroso compound for an addition reaction to occur which produces an electon-spin resonance spectroscopy-detectable aminoxyl radical. In spin trapping, the compound trapping the radical is called the spin trap and the addition product of the radical is identified as the spin adduct”. (Janzen E G., “Spin trapping and associated vocabulary”, Free Rad Res Comm 1990;9(3-6):163, emphasis-added).

Similarly, a Spin Label is “A stable paramagnetic group (typically a nitryl radical) that is attached to a part of a molecular entity whose microscopic environment is of interest and may be revealed by the electron spin resonance (ESR) spectrum of the spin label.” (PAC, 1994, 66, 1077 (Glossary of terms used in physical organic chemistry (IUPAC Recommendations 1994)) page 1166).

The term “nitroxide”, “nitrone”, and “nitroso” are used herein to describe molecules comprising an oxygen and a nitrogen atom directly bound to each other. These compounds may be a electron donors or acceptors. Depending on their oxidation state, these compounds may comprise stable nitroxyl free radicals including precursors (such as the N--H form), and derivatives thereof including their corresponding hydroxylamine derivative (N--OH), where the oxygen atoms are replaced with a hydroxyl group and/or exist in a hydrogen halide form. Nitroxides and nitrones of the invention may be administered to a system, such as a human, and act to modulate oxidation and reduction reactions by donating or accepting an electron. Other mechanisms may include formation of charge-transfer complexes as well as by “redox signaling” or modulation of redox-signaling-mediated processes. Stability of unpaired electrons on such compounds is typically-provided at the nitrogen nucleus by two adjacent carbon atoms that may be substituted with strong electron donor groups. With the partial negative charge on the oxygen of the N--OH bond, the two adjacent carbon atoms together localize the unpaired electron on the nitrogen nucleus. Nitroxides and nitrones generally may have either a heterocyclic or a linear structure. In an vivo environment a nitroxide may react with a first superoxide to form oxoammonium (as an electron donor) and then react with a second superoxide to re-form the nitroxide (as an electron acceptor). (Review: Wilcox C S. Effects of tempol and redox-cycling nitroxides in models of oxidative stress. Pharmacol Ther. 2010 May;126(2):119-45.)

The terms “treat,” “treatment” and the like are used herein to generally mean obtaining a desired pharmacological and/or physiological effect in humans or other animals. A treatment is an approach for obtaining beneficial or desired clinical results. While the claims are not dependent on any specific mechanism, in the present case, these clinical results include but are not limited to decreasing undesirable effects of reactive oxygen species (ROS) and oxidative stress in general, as well as modulating more specific messenger processes such as “redox signaling”. The effect may be therapeutic in terms of a partial or complete cure of the disease and/or adverse effect attributed to the disease. In general, methods of the invention may be applied to a variety of different areas including the skin, mucus membranes, including those in the GI tract, nose, throat, mouth, vaginal cavity, ocular surfaces, as well as the surfaces of the lungs and the surfaces of the vascular system as well as by means of intravenous, intraocular, intramuscular, transdermal, subcutaneous, sublingual, and/or intraoral administration. “Treatment” as used herein covers any treatment of such a symptom or disease in a mammal, particularly a human, and includes:
(a) inhibiting the disease, i.e. arresting it's development; or
(b) relieving the disease and/or it's symptom, i.e. causing regression of the disease and/or the symptoms caused by the disease.

Exemplar Nitrone and nitroxide spin traps and spin labels include, but are not limited to, DEPMPO (5-(Diethoxyphosphoryl)-5-methyl-1-pyrroline N-oxide), TEMPO (2,2,6,6-tetramethyl-1-piperidinyl-1-oxyl), 4-Amino-TEMPO, 4-hydroxy-TEMPO (TEMPOL), DMPO (5,5-dimethylpyrroline-N-oxide), EMPO (2- Ethoxycarbonyl-2-methyl-3,4-dihydro-2H-pyrrole-1-oxide), POBN (alpha-(4- pyridyl-1-oxide)-N-tert-butylnitrone), TEMPONE (4-Oxo-2,2,6,6- tetramethylpiperidine-1-oxyl 4-Oxo-TEMPO), TMIO, 3,3,5,5 tetramethyl-1- pyrolline-N-oxide (TMPO), M3PO (2,5,5-trimethyl-1-pyrroline-N-oxide), M4PO (3,3,5,5-tetramethyl-1-pyrroline-N-oxide), TMPO (3,3,5,5 tetramethyl-1-pyrolline-N-oxide), PBN (1-alpha-phenyl-tert-butyl nitrone), and MNP (2-methyl-2-nitrosopropane, the chief hydrolysis product of PBN), as well as their corresponding hydroxylamine derivatives. The various sulfone (e.g., NXY-059, disulfonyl PBN), hydroxyl, and other derivatives such as esters, peptides, hydroxyl, hydroxylamines, nitrones, carboxyls, and so forth are also claimed.

Preferred examples of the type of hydroxylamine compounds suitable for use in the present invention are TEMPOL-H ((the hydroxylamine reduced form of the nitroxide 4-hydroxy-2,2,6,6-tetramethylpiperidin-1-yloxy), TEMPO-H (the hydroxylamine reduced form of the nitroxide 2,2,6,6-tetramethylpiperidin-1-yloxy) and OXANO-H (2-ethyl-2,4,4-trimethyloxazolidine, which is the reduced form of oxano, 2-ethyl-2,4,4-trimethyloxazolidin-3-yloxy). Other hydroxylamine compounds suitable for use in the present invention include, but are not limited to, those disclosed by Hahn et al. (1998, supra; 2000, supra), Samuni et al. (2001, supra); and in U.S. Pat. No. 5,981,548 to Paolini, et al. (disclosing certain N-hydroxylpiperidine esters and their use as antioxidants in a number of contexts);

U.S. Pat. No. 4,404,302 to Gupta et al. (disclosing the use of certain N-hydroxylamines as light stabilizers in plastics formulations); U.S. Pat. No. 5,462,946 to Mitchell et al. (disclosing certain nitroxides deriving from substituted oxazolidines for protection of organisms from oxidative stress); U.S. Pat. No. 3,936,456 to Ramey et al. (disclosing substituted piperazine dione oxyls and hydroxides for the stabilization of polymers); U.S. Pat. No. 4,691,015, to Behrens et al. (describing hydroxylamines derived from hindered amines and the use of certain of them for the stabilization of polyolefins); and the hydroxylamine compounds disclosed in the several aforementioned U.S. patents to Hsia et al. Most of the above-referenced compounds have not been known heretofore for administration to humans. Certainly, none of them has been known for use in the treatment of fibrocystic disease of the breast.

Suitable reducing agents include, but are not limited to: ascorbic acid, lipoic acid, cystine, purines and derivatives such as acetylcysteine, uric acid and other oxyxanthines, methionine, homocysteine, NADPH, NADH, and so forth.

### EXAMPLES

### Preparation of a Therapeutic Solution of Reduced TEMPOL

The reduced form of TEMPOL (TEMPOL-H) or equivalent pharmacologically-active spin label/spintrap is prepared by mixing together 2 grams of TEMPOL or a pharmacologically effective amount of another spin-label or spin trap such as methynitrosopropane, 20 grams of ascorbic acid or other reducing compound in 100 ml of distilled water. The formulation is used as is. The solution is slightly-bitter. The formulation can be administered diluted in a suitable liquid such as juice, tea, or coffee. An equivalent dry form as 10 mg of tempol mixed with 100 mg of ascorbic acid or equivalent reducing substance can be easily prepared in capsule or tablet form.

### Treatment of Peri-and Post-Menopausal Symptoms

One-half ml of the above solution, comprising 10 mg TEMPOL plus 100 mg ascorbic acid, was administered orally 1-3 times a day to a 53-year old 50 kg woman with perimenopausal symptoms, including hot flashes, sweating, and discomfort. Patient had previously been administered TEMPOL-H for fibrocystic disease of breast, during which time she had not presented with perimenopausal symptoms, despite having stopped menses, but had ceased treatment for several weeks.

First results appeared after 1-2 weeks of treatment and include disappearance of hot flashes, sweating, and discomfort. Patient has also yet to develop the skin changes typical of menopause. Long-term relief can be achieved, but the symptoms eventually reappear on cessation of treatment. Upon resumption of treatment, symptoms disappear.

Alternate forms of oral administration such as tablets, pills or capsules are also effective. Parental modes of administration such as intravenous, intramuscular, subcutaneous, intraperitoneal, transrectal and so forth are also possible, as are topical modes of administration such as in lotions, creams, gels, and topically-compatible suspensions and solutions. Tempol itself is effective at the same doses, but may have increased side-effects at higher doses, e.g., due to oxidation of reducing agents or production of hydrogen peroxide. A 100 mg dose per day of Phenylbutylnitrone is also effective.

Such methods that are routine in the art, and may vary with the needs of individual subjects.

The present invention is not limited to the embodiments described and exemplified above, but is capable of variation and modification within the scope of the appended claims. Also, the claims are not bound by any suggested possible mechanism of action and are independent thereof.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention. Likewise, each claim and indication stands independent of the patentability or patent status of any other claim and indication.

What is claimed is:

1. A method for the treatment of perimenopausal and post-menopausal symptoms in persons in need thereof with a spin trap or its reduced forms at a pharmacologically-effective dose

2. A method for the treatment of perimenopausal and post-menopausal symptoms in persons in need thereof with a spin-label or its reduced forms at a pharmacologically-effective dose.

3. A method for the treatment of perimenopausal and post-menopausal symptoms in persons in need thereof where the spin label is TEMPO (2,2,6,6-tetramethyl-1-piperidinyl-1-oxyl) or a pharmacologically-active derivative. These include 4-hydroxy-TEMPO (TEMPOL), 4 amino-TEMPO, Mito-TEMPOL and/or reduced derivatives such as TEMPO-H or TEMPOL-H, as well as their pharmacologically-active esters, ethers or amides.

4. A method for the treatment of perimenopausal and post-menopausal symptoms in persons in need thereof where the spin-trap is phenylbutylnitrone or 2-methyl, 2-nitrosopropane or their reduced forms.

5. A method for the treatment of perimenopausal and post-menopausal symptoms in persons in need thereof where the pharmacologically-effective dose is 0.01-2000 mg per day of a pharmacologically-active nitroxide, nitrone, or nitroso spin-trap, spin label or their equivalent hydroxylamine or other reduced derivatives administered by suitable means such as orally, sublingually, transrectally, dermally, intramuscularly, or intravenously.

6. A method for the treatment of perimenopausal and post-menopausal symptoms in persons in need thereof where the spin trap or spin label is administered along with a reducing agent.
